(19) United States
(12) Patent Application Publication
(10) Pub. No.: US 2011/0166664 A1
(43) Pub. Date: Jul. 7, 2011

(54) SOCKET HAVING PHYSIOLOGICAL LOAD TRANSMISSION

(75) Inventors: Daniel Delfosse, Jegenstorf (CH); Reto Lerf, Langendorf (CH)

(73) Assignee: Mathys AG Bettlach, Bettlach (CH)

(21) Appl. No.: 13/003,678

(22) PCT Filed: Jun. 2, 2009
(86) PCT No.: PCT/EP2009/003924

§ 371 Date: Feb. 4, 2011

(30) Foreign Application Priority Data
Jul. 11, 2008 (DE) 10 2008 032 705.0
Sep. 12, 2008 (DE) 10 2008 047 009.0

(51) Int. Cl. A61F 2/32

(52) U.S. Cl. 623/22.15

(57) ABSTRACT

The invention relates to a joint socket (1) for a hip-joint endoprosthesis (2), wherein the joint socket (1) comprises ageing-resistant materials and forms a low-abrasion sliding pair together with a joint head (3). The joint socket (1) provides an implant surface (4), which is formed from a material with a porous surface, wherein the isoelastic structure of the joint socket (1) according to the invention and the joint head (3) achieves a physiological load transfer by means of specially designed materials, of which the modulus of elasticity is adapted to the values of a spongy bone material.

The invention relates to a joint socket for a hip-joint endoprosthesis for a total or partial prosthetic treatment of a human or animal hip joint.

European Patent Application EP 1 728 489 A1 describes a hip-joint endoprosthesis with a joint socket which provides blind boreholes. The one-piece joint socket is anchored with at least one screw in the pelvic bone, wherein the screw is arranged in the borehole.

The German published specification DE 43 37 936 A1 describes a spherical hip-joint socket for insertion into bone tissue with an outer metallic shell and an inner ceramic shell which is anchored in the metallic shell.

A hip-joint socket with a coupling element between the socket housing and the socket insert is known from the German published specification DE 199 04 436 A1, wherein the elasticity and damping property of the coupling element can be predetermined through its porosity and through the structure of its surface.

The hip-joint endoprostheses described in the prior art have in fact attained a high standard, wherein a survival rate of at least 90% after 10 years has been achieved, that is to say, a dropout rate of 1% per year has not been exceeded. As has been demonstrated with reference to the Scandinavian hip register, there are even systems (in each case the best cemented or un-cemented hip shafts or sockets in their class), which provide survival rates after 10 years of 95% or more. However, in the second decade after the implantation, the dropout rate increases dramatically. Accordingly, there are currently no hip joint sockets, which still provide a survival rate of 90% or more even after 20 years.

Four reasons can be given for this:
Not all of the materials which have been used up to the present with a total hip-joint replacement guarantee that they will remain “un-aged”, that is without significant impairment of their mechanical and tribological properties, over very long periods in the body.
Not all of the materials which have been used up to the present as articulation partners in a total hip-joint replacement guarantee that the abraded particles behave benignly over very long periods in the body without causing tissue damage.
Not all of the materials which have been used up to the present on the surface of the total prosthetic treatment of the hip joint guarantee that the bone surrounding the implant connects with the implant in the long-term and permanently, that is to say, in an “osseointegrated” manner.
Not all of the materials which have been used up to the present as load-bearing structure for the total prosthetic treatment of the hip joint guarantee that the bone surrounding the implant will not recede during the course of time as a result of so-called “stress shielding”.

The invention is based on the object of providing a joint socket for a prosthetic treatment of a human or animal hip joint which is still firmly anchored in the body even in the second decade after the implantation and which fulfils the following conditions:
The material for the joint socket should remain un-aged over more than two decades in the body.
The material of the joint socket should be abrasion-resistant over a very long period.
The material on the surface of the joint socket should connect in a permanent and stable manner with the surrounding bone material.
The material of the joint socket should introduce the mechanical load into the surrounding bone material in a uniform manner.

With regard to all of the conditions listed above, the object according to the invention is achieved by the features of claim 1. Advantageous further embodiments form the subject matter of the dependent claims referring back to claim 1.

Claim 1 describes a joint socket for a hip-joint endoprosthesis, wherein the joint socket according to the invention comprises ageing-resistant materials and forms a low-abrasion sliding pair together with a joint head. The joint socket according to the invention provides an implant surface, which is formed from a material with a porous surface. Accordingly, an isoelastic structure of the joint socket according to the invention forms a physiological load transfer together with the natural or implanted joint head, wherein specially designed materials are used, of which the modulus of elasticity is adapted to the values of a spongy bone material.

One advantage of the joint socket according to the invention is that the material for the joint socket is a composite material with a modulus of elasticity preferably between 0.3 GPa and 2.0 GPa, by particular preference between 0.5 GPa and 1.5 GPa.

The composite material of the joint socket according to the invention advantageously guarantees a physiological load transfer to the surrounding bone structures. If the implants are more rigid than the bone replaced, a large part of the load is taken up by the implant and generally transferred again to a position in the surrounding bone remote from the articulation. A bone close to the articulation, such as the proximal femur, is therefore insufficiently loaded and atrophies as a result. All materials with a high modulus of elasticity are unsuitable. The absolute upper limit is considered to be 17 GPa, which corresponds to the average modulus of elasticity of the cortical bone. However, in order to achieve an optimum bone integration of the joint socket according to the invention, this must, as far as possible, not change the mechanical properties of the pelvis. The ideal material for a pelvic joint socket is adapted with regard to the modulus of elasticity to the corresponding values of the spongy bone (0.3 GPa-2.0 GPa). Accordingly, the mechanical, and in particular the deformation, behaviour of the pelvis is advantageously not influenced.

For the manufacture of the joint socket according to the invention, a composite material is advantageously used so that the modulus of elasticity always falls significantly below the absolute upper limit of 17 GPa.

Furthermore, it is favourable that the composite material comprises a given proportion of a polymer, which is easy to form. A reinforcement of the polymer with a second material, which can be armour-plating, fibres or particles, is expediently provided. The second material is advantageously a metal, a ceramic or a second polymer, which connects well to the first polymer or respectively can be readily integrated into the latter.

Furthermore, it is advantageous if the joint socket according to the invention provides an implant surface with particles of titanium or calcium phosphate. Accordingly, the surface is additionally enlarged, which promotes good osseointegration.

A further advantage of the joint socket according to the invention is preferably in the manufacture of its surfaces from inert or bio-active materials, such as titanium or hydroxyl apatite. Accordingly, an enduringly strong connection with the surrounding bone is achieved through osseointegration, wherein the surfaces are advantageously porous or deeply roughened.

A further advantage of the joint socket according to the invention is its preferred implant surface of titanium, tantalum, calcium phosphate or bio-glass, which is also favourable for osseointegration.

Furthermore, it is advantageous if the joint socket according to the invention contains stabilised and highly cross-linked UHMWPE which is resistant to ageing and oxidation through an addition of an antioxidant and does not become brittle, thereby guaranteeing that very few if any abrasion particles are generated over a period of more than two decades.

The antioxidant is preferably vitamin E, which can easily be added in a metered manner as a liquid before or after the sintering of the UHMWPE powder. These methods are described in EP 1 161 489 A1, WO 2004/101009 A1 and WO 2004/064618 A1.

Furthermore, a joint socket manufactured from a monolithic material is advantageous, because the manufacturing tolerances can be kept small for this type of manufacture.

A joint socket built up as a composite, which provides an implant surface in which a ceramic inlay is integrated inseparably for the user, is advantageous in that the sliding pairing between the joint head and the interior of the joint socket according to the invention is optimised.

Exemplary embodiments of the present invention are described below with reference to the drawings. The drawings are as follows:

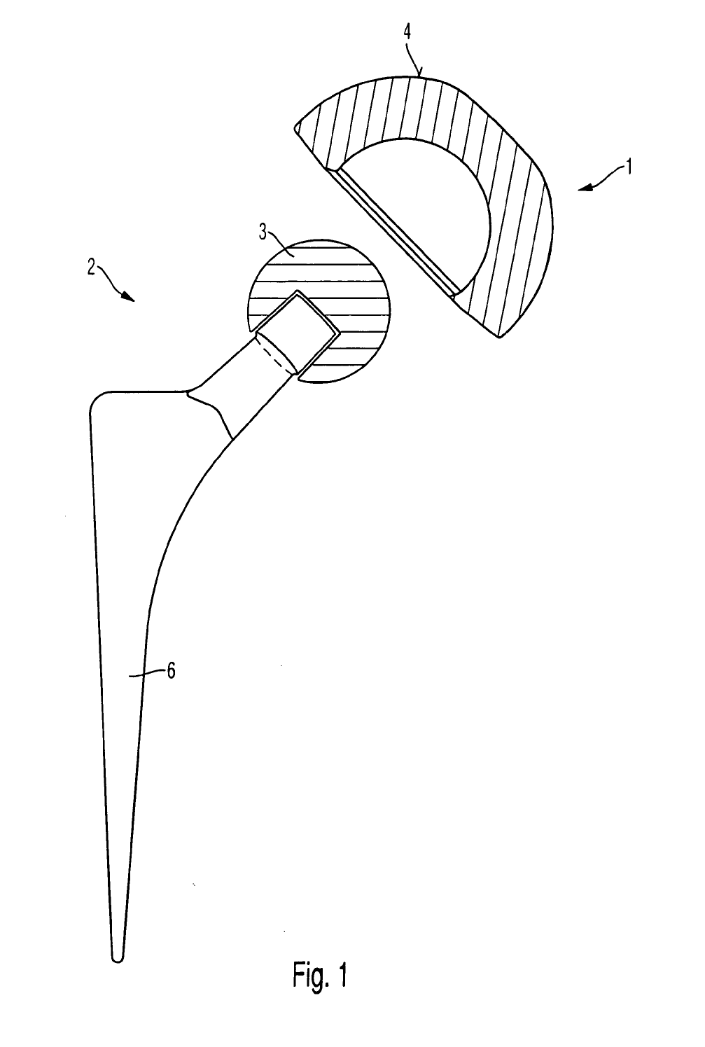

FIG. 1 shows a total hip-joint endoprosthesis with the joint socket according to the invention;

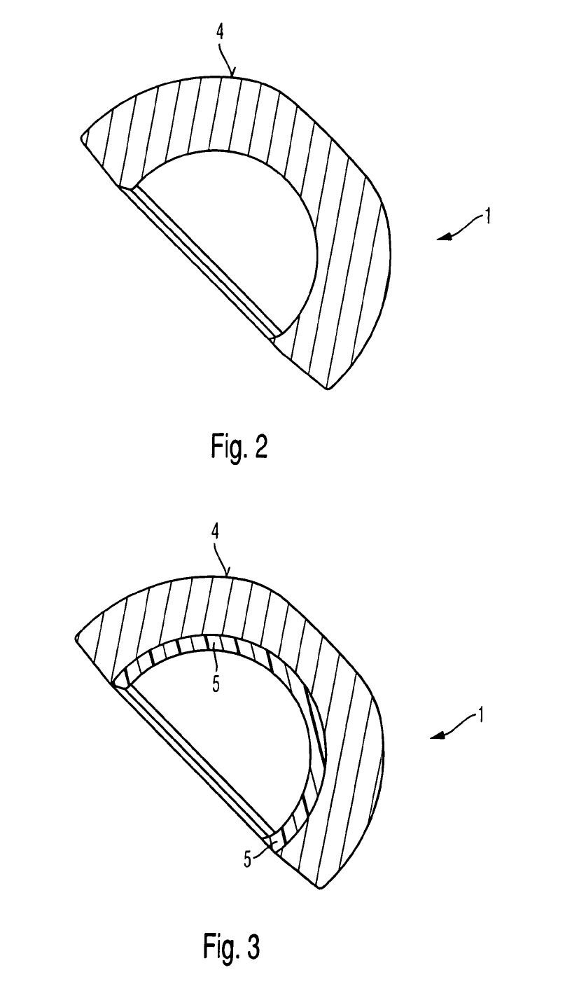

FIG. 2 shows a sectional view of a first exemplary embodiment of the joint socket according to the invention; and

FIG. 3 shows a sectional view of a second exemplary embodiment of the joint socket according to the invention.

Mutually corresponding parts are provided with the same reference numbers in all drawings.

FIG. 1 shows a total hip-joint endoprosthesis 2 with the joint socket 1 according to the invention, which, together with a joint head 3 according to the present invention, forms a low-abrasion and ageing-resistant sliding pair, which can still remain fully functional in the human body even 20 years after its implantation. With a total hip-joint endoprosthesis 2, the joint head 3 is attached to a proximal end of a shaft 6, which is implanted into the bone-marrow channel of a femur bone. In the case of a partial hip-joint endoprosthesis, by contrast, only the joint socket 1 is anchored in the bone material of the pelvis.

FIG. 2 shows a first exemplary embodiment of the joint socket 1 according to the invention for a hip-joint endoprosthesis 2. The joint socket 1 comprises ageing-resistant materials and forms a low-abrasion sliding pair together with a joint head 3, wherein the joint socket 3 according to the invention provides an implant surface 4, which is formed from a material with a porous surface. An isoelastic structure of the joint socket 1 achieves a physiological load transfer by means of specially designed materials by matching their moduli of elasticity according to the invention to the values of a spongy bone material.

The first exemplary embodiment of the joint socket 1 according to the invention shown in FIG. 2 relates to a manufacture from a single monolithic material, so that a precise and cost-favourable manufacture of the joint socket 1 according to the invention is possible by means of milling or turning from a single material block. The isoelastic structure of the joint socket 1 according to the invention is then provided, if its mechanical properties such as strength and loadability correspond to the mechanical properties of the bone material in which the joint socket is implanted.

The material for the joint socket 1 according to the invention is a composite material with a modulus of elasticity between 0.3 GPa and 2.0 GPa, wherein the composite material, which comprises a given proportion of polymer, preferably provides a modulus of elasticity within the range from 0.5 GPa to 1.5 GPa and by particular preference within the range from 0.8 GPa to 1.2 GPa.

For the polymer of the composite material, a reinforcement with a second material can be provided, wherein armour-plating, fibres or particles, which are made from a metal, a ceramic, graphite or a second polymer with a relatively high strength are provided as the reinforcement.

The joint socket 1 according to the invention provides a structured implant surface 4 made of inert or bio-active materials, such as titanium, tantalum, calcium phosphate, hydroxyl apatite or bio-glass, wherein the stabilised UHMWPE provides improved material properties and an increased resistance to ageing and oxidation as a result of an addition of an antioxidant, such as a vitamin E. In this context, the vitamin E is supplied before or after the sintering of the UHMWPE powder to form a solid.

The implant surface 4 of the joint socket 1 according to the invention can comprise porous titanium, tantalum, calcium phosphate or bio-glass or can comprise individual particles of titanium or calcium phosphate, which additionally improve the osseointegration into the bone material without substantially increasing the stepping of the composite.

FIG. 3 shows a second exemplary embodiment of the joint socket 1 according to the invention with a modular structure, wherein a ceramic inlay 5, which forms an ageing-resistant and very low-abrasion sliding pair together with the physiological or implanted joint head 3, is integrated into the implant surface of the joint socket 1 according to the invention.

The ceramic inlay 5 is designed in such a manner that it does not substantially reduce the elasticity of the socket (for example, measured as radial stepping [??] over the external equatorial line). Conversely, the wall thickness of the ceramic inlay 5 must not fall below a given minimum value, in order to guarantee security against fracture. The thickness is disposed at around 2 to 4 mm, dependent upon the ceramic (by preference a dispersion ceramic is used).

The invention is not restricted to the exemplary embodiments presented in the drawings, especially not to a total prosthetic treatment of a human or animal hip joint. An application of the joint socket 1 according to the invention in a shoulder prosthesis is also provided. All of the features described and presented in the drawings can be combined with one another as required.

1. A joint socket for a hip joint endoprosthesis which, together with a joint head, forms a sliding pair, wherein the joint socket provides an implant surface, which is formed from a material with a porous surface, and wherein an isoelastic structure of the joint socket in cooperation with the joint head achieves a physiological load transfer by means of a material for the joint socket,
wherein a modulus of elasticity of the material for the joint socket is adapted to the values of a spongy bone material and is between 0.3 GPa and 2.0 GPa.

2. The joint socket according to claim 1, wherein the material for the joint socket is a composite material with a modulus of elasticity between 0.5 GPa and 1.5 GPa.

3. The joint socket according to claim 2, wherein the composite material for the joint socket provides a modulus of elasticity within the range from 0.8 GPa to 1.2 GPa.

4. The joint socket according to claim 2, wherein the composite material comprises a given proportion of a polymer.

5. The joint socket according to claim 4, wherein a reinforcement with a second material is provided for the polymer.

6. The joint socket according to claim 5, wherein the reinforcement is provided by armour-plating, fibres or particles selected from material consisting of a metal, a ceramic, graphite and a second polymer of a relatively greater strength.

7. The joint socket according to claim 1, wherein the implant surface comprises inert or bio-active materials selected from the group consisting of titanium, tantalum, hydroxyl apatite, calcium phosphate and bio-glass.

8. The joint socket according to claim 7, wherein the composite material contains stabilised UHMWPE, wherein the stabilised UHMWPE is ageing-resistant and oxidation-resistant by means of an addition of an antioxidant.

9. The joint socket according to claim 8, wherein the antioxidant supplied to the UHMWPE is vitamin E.

10. The joint socket according to claim 9, wherein powdered vitamin E is supplied before, during or after the sintering polymerisation of the UHMWPE powder.

11. The joint socket according to claim 1, wherein the joint socket comprises a monolithic material.

12. The joint socket according to claim 11, wherein the implant surface provides particles of titanium or calcium phosphate.

13. The joint socket according to claim 1, wherein the joint socket defines a modular structure and the implant surface comprises a ceramic inlay integrated therein.

14. The joint socket according to claim 13, wherein the implant surface provides particles of titanium or calcium phosphate.
